(19) United States
(12) Patent Application Publication
(10) Pub. No.: US 2005/0227918 A1
(43) Pub. Date: Oct. 13, 2005

(54) Method for assaying susceptibility to cancer therapy in a subject by assaying for tumors of P53

(76) Inventors: Paul J. Farrell, London (GB); Xin Lu, London (GB); Tim Crook, London (GB)

Correspondence Address: FULBRIGHT & JAWORSKI, LLP, 666 FIFTH AVE, NEW YORK, NY, 10103-3198, US

(21) Appl. No.: 10/820,482

(22) Filed: Apr. 7, 2004

(51) Int. Cl. A61K 38/17; G01N 33/574; C12Q 1/68

(52) U.S. Cl. 514/12; 435/7.23; 435/6

(57) ABSTRACT

The invention relates to methods for determining a subject's susceptibility to cytotoxic drug therapy, by determining what polymorphic form of p53 the subject expresses. Also, a part of the invention are methods for identifying susceptible subjects, by assaying for mutated forms of p53. Therapeutic approaches based on this are also contemplated.

### FIELD OF THE INVENTION

This invention relates to methods for determining whether or not a cancer patient is a suitable subject for chemotherapy. It also relates to the methods for treating such patients.

### BACKGROUND AND PRIOR ART

Apoptosis is a complex, basic cellular mechanism by which, e.g., DNA damaging, anticancer agents lead to cytotoxicity. See Johnstone, et al., Cell, 108:153-164 (2002), for an example of the literature on this area.

To elaborate, however, it is now clear that p53 has a major role in transducing stress to the apoptotic machinery of the cell, a role that is consistent with the molecule's importance in determining the cellular response to DNA damaging drugs. See Lowe, et al., Cell, 75:957-967 (1993); Lowe, et al., Science, 266:807-810 (1994).

Following exposure to cellular stress, the DNA-binding function of p53 is “activated,” and leads to cell cycle arrest, senescence or apoptosis. See Vogelstein, et al., Nature, 408:307-310 (2000). Activation of p53 involves post-translational modifications such as phosphorylation and acetylation. After such modifications, the protein is stabilized and acquires increased DNA-binding affinity.

Several p53 target genes have been described. See el-Diery, Semin. Cancer Biol., 8:345-357 (1998). These include genes involved in cell cycle arrest such as p21^{waf1} and 14-3-3σ, as well as genes involved in mediating apoptosis such as AIP1, Bax, PUMA, Noxa and PIGPC1. See Vousden and Lu, Nat. Rev. Cancer, 2:594-604 (2002).

It is also known that the presence of intact p53 function acts to confer tumor sensitivity to DNA damaging agents, such as cisplatin. This, in turn, leads to therapeutic potential for, e.g., germ-cell tumors. See Riou, et al., Mol. Carcinog., 12:124-131 (1995).

One mechanism through which DNA-damaging anticancer agents exert their biological effects is p53-dependent apoptosis. See Lowe, et al., Cell, 75:957-967 (1993); Lowe, et al., Science, 266:807-810 (1994); Johnstone, et al., supra. Serine 46 (46S) is reported to function in the regulation of p53-dependent apoptosis, via pro-apoptotic genes such as AIP1. See Oda K., et al., Cell, 102:849-862 (2000). However, AIP1 expression is upregulated only at later stages of apoptosis and at high levels of DNA damage. Chromatin immunoprecipitation (ChIP) assays have questioned the likelihood of this gene as being a direct effector of p53-dependent apoptosis. See Kaeser and Iggo, Proc. Natl. Acad. Sci. USA, 99:95-100 (2002).

It seems that PUMA (Nakano and Vousden, Mol. Cell., 7:683-694 (2001); Yu, et al., Mol. Cell., 7:673-682 (2001)) and Noxa (Oda E., et al., Science, 288:1053-1058 (2000)) may be more likely candidates for affecting p53-dependent apoptosis. Cells lacking these proteins are defective in apoptosis, at least in response to some cellular stresses. See Shibue, et al., Genes Dev. 17:2233-2238 (2003); Villunger, et al., Science, 302:1036-1038 (2003). Apoptosis induction is the function of p53 selected against in tumorigenesis (Schmitt, et al. Cancer Cell, 1:289-298 (2002)). Accordingly, almost all human tumor-associated p53 mutants are defective for apoptosis.

A single-nucleotide polymorphism (SNP) in exon 4 of p53 results in the presence of either arginine (R) or proline (P) at codon 72. See Matlashewski, et al., Mol. Cell. Biol., 7:961-963 (1987). The polymorphism is balanced, varies with latitude and race, and is maintained at different allelic frequencies across the population of the world. See Sjalander, et al., Hum. Hered., 45:144-149 (1995). Further the polymorphism is located in the proline-rich domain, which is important in the apoptosis function of p53. See Baptiste, et al., Oncogene, 21:9-21 (2002). Taken together, these observations imply biologically important differences between the 72R and 72P variants, which are subject to natural selection pressures.

There is experimental evidence that the 72R form of p53 possesses greater apoptosis-inducing potential than the 0.72P variant. See Thomas, et al., Mol. Cell. Biol., 19:1000-1092 (1999); Dumont, et al., Nat. Genet., 33:357-365 (2003); Pim and Banks, Int. J. Cancer, 108:196-199 (2004). Mechanistically, this has been linked to differential nuclear/cytoplasmic transport of the 72R and 72P forms and localization at the mitochondrian. See Dumont, et al., supra.

The great majority of cells in the body contain wild-type p53, which mediates the normal physiological function of p53. Wild-type p53 can contain either the 72R or 72P variant. The following examples show that the 72R and 72P forms of wild-type p53 differ in their ability to cause apoptosis following exposure to drugs from distinct mechanistic classes of agent. This is linked, at least in part, to the differential transcriptional effects of p53.

While p53 does have a role in determining cellular sensitivity to anticancer agents, cancers with intact p53 function are a minority. Mutations in p53 are known to occur in over 50% of human cancers. See, e.g., Vogelstein, et al., Nature, 408:307-310 (2000). Mutant forms of the protein are generally compromised for apoptotic induction. Schmitt, et al., supra, provide evidence that during tumorigenesis, p53 function is selected against.

Whereas wild type p53 function is compromised in the majority of human tumors, it is intact in normal cells of the same hosts. See Vogelstein, et al., supra. This means that the selectivity of anticancer drugs cannot be accounted for by p53 apoptosis alone.

A relative of p53, i.e., p73, also functions in transduction of specific types of DNA damage. Activation of the p73/c-abl pathway by certain therapeutic agents causes apoptosis via genes such as AIP1. See, e.g., Gong, et al., Nature, 399:806-809 (1999); Costanzo, et al., Mol. Cell., 9:175-186 (2002). Various splice variants of p73 exist, with modifications at both the N and C termini. Stiewe, et al., Cancer Res., 62:3598-3602 (2002), show that N-terminal variants which lack the transactivation domain may act as dominant-negative inhibitors of both p53 and p73.

In squamous cell carcinomas, i.e., “SCCs”, mutants occur in the 72R allele of wild type p53 more commonly than the 72P allele. See, e.g., Marin, et al., Nat. Genet., 25:47-54 (2000); Brooks, et al., Cancer, 60:6875-6877 (2000). Others have verified these findings, in other cancers. See Tada, et al., Carcinogenesis, 22:515-517 (2001); Furihata, et al., Clin. Cancer Res., 8:1192-1195 (2002).

Nonrandom retention and expression of p53 mutants implies that there are selective pressures during tumorigenesis that confer selective advantages to cancer cells which express 72R mutants, resulting in over representation of these mutations in carcinomas. It is also known that p73 and related molecules p63 are targets for transdominant inhibition by p53 mutants. See Di Como, et al., Mol. Cell Biol., 19:1438-1449 (1999); Marin, et al., supra; Strano, et al., J. Biol. Chem., 275:29503-29512 (2000); Strano, et al., J. Biol. Chem., 275:18817-18826 (2002); Galddon, et al., Mol. Cell Biol., 21:1874-1887 (2001).

One mechanism which may account for the bias toward 72R mutants was suggested by the observation that inhibition of p73 is influenced by the polymorphism at position 72. Indeed, two mutations of the 72R form, i.e., 143A and 175H, associate more efficiently with p73, and inhibit p73 dependent target gene regulation than do equivalent, 72P mutants. See Marin, et al., supra. It has also been observed, however, that other mutants associate with p73, in both polymorphic forms. See Marin, et al., supra, Gaiddon, et al., supra, Soussi, et al., Nat. Rev. Cancer, 1:233-239 (2001), suggest that treatment outcome in patients may be influenced by p53 polymorphism.

In the disclosure that follows, it is now shown that, in fact, treatment outcome can be correlated to polymorphisms in the p53 wild type alleles, (i.e., 72R and 72P). This will be seen in the disclosure which follows.

### EXAMPLE 1

The following experiment was conducted to determine whether the reported differences in the apoptosis-inducing potential of the two polymorphic forms of wild-type p53 result in distinct cellular responses to clinically important anticancer drugs.

72R and 72P wild-type p53 plasmids were constructed by subcloning the inserts from pArgSP53 and pProSP53 (See Matlashewski, et al., Mol. Cell. Biol., 7:961-963 (1987), incorporated by reference) into commercially available pTRE, resulting in pTRE 72R p53 and pTRE 72P p53 plasmids. Human H1299 p53−/− cells, which were maintained in DMEM+10% fetal bovine serum, were transfected with the pTRE 72R p53 and pTRE 72P p53 plasmids in accordance with standard procedures, resulting in H1299 cells lines which inducibly express 72R or 72P p53 protein. These cell lines were grown in DMEM 10% FCS supplemented with 0.5 mg/ml G418 and 0.5 μg/ml puromycin. Expression of 72R or 72P wild-type p53 was induced in H1299 cells by doxycycline or non-induced. After 18 hours, the cells were then exposed to cisplatin (1 μ/ml), taxol (500 ng/ml), etoposide (10 μM) or doxorubicin (1 μg/ml). Control cells were not exposed to drugs. Flow cytometric analysis was then performed after 24 hours as described in Bergamaschi et al., Cancer Cell, 3:387-402 (2003), incorporated by reference. A minimum of six independent 72R-inducible and 72P-inducible clones were analyzed, to obviate the effect of clonal variation. Western blotting of the cells 24 hours after exposure to the drugs demonstrated equal expression of 72R and 72P proteins.

The results showed that when induced cells were exposed to anticancer agents, apoptosis was always higher in cells expressing the 72R wild-type p53 variant, while the predominant effect in cells expressing the 72P wild-type p53 variant was G1 arrest. For each agent tested, the sub-G1 (apoptotic) fraction was higher in cells expressing 72R p53. This was most clearly seen in cells treated with taxol and cisplatin, where induction of 72P p53 caused minimal apoptosis but predominantly G1 arrest, whereas induction of 72R p53 resulted in an increased sub-G1 fraction and a smaller G1 peak. In the absence of drug treatment, expression of either 72R or 72P p53 resulted in only low levels of apoptosis. Similarly, exposure of non-induced cells to the same agents caused only a small (but reproducible) increase in sub-G1 cells compared to untreated cells.

Further tests examined the cellular responses to the anticancer agents camptothecin and 5-fluorouracil (FU). H1299 cells were induced to express equal levels of 72R or 72P wild-type p53, as described supra, or not induced. Equal expression levels of 72R and 72P p53 were confirmed by Western blotting. The cells were then exposed to camptothecin (5 μM) or 5-FU (50 μg/ml). Flow cytometric analysis was performed after 24 hours. Again, when induced cells were exposed to these agents, apoptosis was always higher in cells expressing the 72R p53 variant, whereas the predominant effect in cells expressing the 72P p53 variant was G1 arrest.

### EXAMPLE 2

The following experiments were performed to reproduce the differences in cell cycle distribution and apoptosis demonstrated in Example 1.

Three experiments were conducted, each analyzing two independent clones of 72R-expressing H1299 cells and two independent clones of 72P-expressing H1299 cells. The clones were either treated with doxycycline to induce p53 to similar steady-state levels, or not induced. The cells were then exposed to cisplatin (1 μg/ml), taxol (500 ng/ml), etoposide (10 μM), doxorubicin (1 μg/ml), camptothecin (5 μM) or 5-FU (50 μg/ml). Control cells were not exposed to drugs, and control cells for camptothecin and 5-FU received the drug vehicle only. After 24 hours, apoptosis was determined by flow cytometry as previously described.

The results confirmed that apoptosis is higher in cells expressing 72R wild-type p53 compared to cells expressing 72P wild-type p53. The difference in apoptosis between 72R and 72P was as high as eightfold in the case of doxorubicin, but was never less than twofold, as in the case of camptothecin.

### EXAMPLE 3

This next experiment was conducted to verify that similar effects occur when cells expressing endogenous p53 are exposed to the same anticancer agents.

EBV-immortalized B-lymphoblastoid cell lines were selected for this experiment because they retain a p53-dependent, DNA damage-inducible pathway, and are thus an appropriate system for comparative studies. See Allday, et al., EMBO J., 14:4994-5005 (1995). The cell lines were maintained in RPMI 1640 medium with 10% FBS. EBV-immortalized B-lymphoblastoid cell lines of germ-line genotype 72RR or 72PP were exposed to cisplatin (1 μg/ml), taxol (500ng/ml), etoposide (10 μM) or melphalan (10 μg/ml). Control cells were not treated with drugs. After 8 hours, cells were analyzed by flow cytometry.

Apoptosis was reproducibly higher in cell lines of 72RR genotype compared to those of 72PP genotype. Time course analysis also revealed that the onset of apoptosis was more rapid in the 72RR cells. The 72RR cells had a detectable level of sub-G1 cells after 8 hours for each anticancer agent, whereas no increase in apoptosis was seen in cells expressing 72PP.

Further tests were performed to determine if the differences in apoptosis-inducing activity between 72R wild-type p53 and 72P wild-type p53 were attributable to differences in the kinetics of p53 stabilization. EBV-immortalized B-lymphoblastoid cell lines of germ-line genotype 72RR or 72PP were exposed to etoposide (10 μM). PCNA was used as a control protein. Aliquots of cells were then harvested at 0, 2, 4, 6, 8, 10, and 12 hours and analyzed by Western blotting.

The kinetics of p53 stabilization between the two cell lines were indistinguishable, indicating that the differences in apoptosis-inducing activity seen in the above examples are not attributable to differential stability.

### EXAMPLE 4

This example describes experiments used to determine whether the differences in pro-apoptotic activity between 72R and 72P polymorphic variants of wild-type p53 was reflected in different cytotoxicities of anticancer drugs.

The cytotoxicity of anticancer agents was determined in colony-survival assays of H1299 cells using standard protocols, following Bergamaschi, et al., supra, and as per the earlier examples. Briefly, H1299 cells were treated with doxycycline to induce p53 and plated at multiple densities. Then they were exposed to varying concentrations of cisplatin (0-10.0 μg/ml) or doxorubicin (0-2.0 μg/ml) for 2 hours. After drug exposure, cells were re-plated in drug free medium and grown until the appearance of visible colonies. Each drug exposure was done in triplicate in at least three experiments. The experiments were repeated with etoposide, 5-FU and taxol, yielding similar results.

For each agent tested, cytotoxicity was greater in 72R-expressing cells than in 72P-expressing cells.

### EXAMPLE 5

These experiments were designed to identify the genes differentially expressed between the 72R and 72P proteins.

H1299 cell lines were induced to express 72R or 72P wild-type p53, as described supra, or were not induced. Expression of p53 was determined by Western blotting as previously described. Cells were then treated with cisplatin (1 μ/ml) or the drug vehicle (control). Total RNA was prepared from non-induced cells, and induced cells expressing equal steady-state levels of 72R and 72P p53. All cells were harvested at 0 and 12 hours. This was then used to probe an array of p53 target genes, in accordance with standard methods. Each hybridization was performed at least three times. Differences in upregulation were verified by determining levels of mRNA for p21^{waf1}, PIGPC1, AIP1 and PUMA by quantitative PCR, according to standard procedures.

The mRNA of three genes was induced more efficiently in clones expressing 72R p53 than 72P p53. The genes more efficiently upregulated by 72R were PUMA (BBC3), PIGPC1 (PERP) and AIP1, but there was no difference in induction of p21^{waf1} or MDM2.

In further tests, H1299 cells inducibly expressing 72R or 72P wild-type p53 were exposed to doxorubicin (1 μg/ml), etoposide (10 μM) or taxol (500 ng/ml), or not exposed to drugs (control). Total RNA was prepared from cells harvested at 0 and 12 hours and levels of mRNA for AIP1, PIGPC1 and PUMA were determined by quantitative PCR, according to standard procedures. Three independent clones of H1299 cells expressing 72R wild-type p53 and three expressing 72P wild-type p53 were tested.

Results showed that expression of AIP1, PIGPC1 and PUMA is more efficiently activated by anticancer agents in cells expressing 72R wild-type p53.

### EXAMPLE 6

In the next experiment, CHIP assays were used to verify that the increased induction of PUMA, PIGPC1 and AIP1 was attributable to enhanced specific DNA binding of the 72R protein.

H1299 cell lines were induced to express 72R or 72P wild-type p53, as described supra, and were treated with cisplatin (1 μg/ml). After 24 hours, cell lysates were harvested for CHIP analysis in accordance with Kaeser and Iggo, Proc. Natl. Acad. Sci. USA, 99:95-100 (2002), incorporated by reference. Each assay included a positive control. (Sp1 binding to the DHFR reporter) and a negative control (GAPDH). The p53 was immunoprecipitated by anti-p53 DO-1 antibody at 4° C. for one hour. PCR for the AIP 1, p12^{waf1} MDM2 and PUMA promoters was performed with the primers described in Kaeser and Iggo, supra. Amplification was carried out under the following conditions: 10 minutes at 95° C., 30 seconds denaturing at 95° C., 1 minute annealing at 55° C. and 1 minute extension at 72° C. Probes were synthesized as described in Kaeser and Iggo, supra.

In the case of the p21^{waf1} and MDM2 promoters, there were only small differences in DNA binding between the 72R variant and the 72P variant. This was consistent with PCR analyses described above, demonstrating similar induction of mRNA. In contrast, the 72R variant reproducibly bound the AIP1 and PUMA promoters and activated these genes with higher efficiency than the 72P variant.

### EXAMPLE 7

Next, luciferase assays were conducted to determine whether the promoters of other apoptosis-associated genes were also more efficiently transactivated.

H1299 cells were transfected with pCB6⁺72R or pCB6⁺72P wild-type p53 expression plasmids and commercially available luciferase reporter constructs, together with pSV2β-gal (control for transfection efficiency). Cells were treated, or not, with cisplatin (1 μg/ml) as well. After 24 hours, cells were harvested for determination of luciferase and α-galactosidase activities according to standard methods.

Consistent with ChIP assays performed in Example 6, the PUMA promoter was more efficiently transactivated by 72R. No difference was detected in the activation of either Bax or Bid. In contrast, the Noxa promoter was reproducibly more efficiently activated in H1299 cells by 72R wild-type p53 than by 72P wild-type p53.

### EXAMPLE 8

The experiments described in this example were conducted to determine whether the observed differences between the 72R and 72P variants of wild-type p53 involved residue 46S of p53. 46S was selected for this test because it has been reported to be important in p53-dependent apoptosis. See Oda K. et al., Cell, 102:849-862 (2000). Normally, position 46 of wild-type p53 is alanine.

H1299 cells inducibly expressing 46A 72R or 46A 72P were prepared as described supra. 46S p53 mutants were constructed in the pTRE 72R p53 and pTRE 72P p53 plasmids (described in Example 1) according to standard methods, and verified by sequencing. H1299 cells were cotransfected with these plasmids and pBabePuro to inducibly express 46S 72R or 46S 72P.

These four H11299 cell lines (46A 72R, 46A 72P, 46S 72R, and 46S 72P) were treated with doxycycline to induce expression of the variants or were left untreated. Expression of p53 and PCNA (loading control) was determined by Western blotting, in accordance with standard methods, to define conditions for induction of equal steady-state levels of p53 protein.

Twenty-four hours after induction with doxycycline, lysates were prepared and ChIP analysis was performed, as described supra. Promoters for p21, MDM2, AIP1 and PUMA were used.

There was relatively greater binding of the 46S 72R protein to the AIP1 and PUMA promoters, which was reduced by substitution of 46S with A. There was little difference, however, between the 46S and 46A variants of the 72R and 72P proteins in binding to the p21 and MDM2 promoters.

Further tests were done to analyze the induction of PUMA and AIP1 mRNA by 72R and 72P wild-type p53. H1299 cells were treated with doxycycline to induce the expression of the 46A 72R, 46A 72P, 46S 72R, or 46S 72P p53 variants. Next, the cells were exposed to either cisplatin (1 μg/ml) or drug vehicle (control). After 24 hours, RNA was prepared according to standard methods, and levels of PUMA and AIP1 were determined by quantitative. PCR. This experiment was conducted twice, each analyzing two independent clones of each p53 variant.

The results showed that p53-dependent induction of PUMA and AIP1 was reduced in cells expressing the 46A variants of wild-type p53.

### EXAMPLE 9

Next, apoptosis was measured in cells expressing the 46A and 46S variants of 72R and 72P wild-type p53.

H1299 cells were cotransfected with pcDNA3 CD20 and plasmids expressing 46A 72R, 46A 72P, 46S 72R, or 46S 72P wild-type p53 or with an empty vector (no p53, as a control), as described supra. The cells were then treated with cisplatin (1 μg/ml). After 24 hours, the cells were analyzed by flow cytometry. Western Blot analysis of p53 expression was also performed.

Apoptosis in cells expressing 46A 72P p53 was marginally reduced relative to 46S 72P p53; however, apoptosis in cells expressing 46A 72R p53 was reduced to a level comparable to that in cells expressing 46S 72P p53 and 46A 72P p53. The Western Blot showed that levels of p53 protein increased after cisplatin treatment for each of the four variants, indicating that the effect on apoptosis was not related to differences in the stability of the proteins after DNA damage.

Follow-up experiments were conducted to determine if these results were reproducible. H1299 cells were either treated with doxycycline to induce expression of the 46A 72R, 46A 72P, 46S 72R, or 46S 72P variants of p53 or were untreated (control). After 18 hours, the cells were exposed to cisplatin (1 μg/ml), doxorubicin (1 μg/ml), etoposide (10 μM), 5-FU (50 μg/ml) or were exposed to drug vehicle only (control). Cells were analyzed by flow cytometry 24 hours later.

The results showed that 46S influences the induction of apoptosis in 72R-expressing cells.

Further tests were performed to see if 46S influences cellular sensitivity to anticancer drugs in 72R-expressing cells. Cytotoxicity of cisplatin and doxorubicin was determined in colony-survival assays of H1299 cells, as described supra. Consistent with the apoptosis assays, the cytotoxic effect of cisplatin and doxorubicin was diminished in cells expressing 46A 72R p53 relative to 46S 72R p53, but the sensitivity of cells expressing 46A 72P p53 was similar to that of cells expressing 46S 72P p53.

These experiments show that p53-mediated apoptosis occurs in the absence of 46S, but indicates that the relatively greater apoptosis-inducing and cytotoxicity-inducing activity of the 72R protein is dependent, at least in part, on the presence of 46S.

### EXAMPLE 10

The following experiments were conducted to investigate the possibility that polymorphism in wild-type p53 may affect response and outcome in cancer treatment by virtue of its effect on drug-induced apoptosis.

Tissue samples were obtained from 70 patients with inoperable, advanced head and neck squamous cell carcinomas (HNSCC) who received cisplatin-based chemo-radiotherapy. All patients presented with locally advanced, unresectable, TNM stage III/IV HNSCC. Tissues were obtained as paraffin sections or frozen tissues from patients undergoing diagnostic biopsy prior to commencement of chemo-radiotherapy. The diagnosis and presence of an adequate proportion of tumor tissue in each sample was confirmed by histopathological analysis, in accordance with standard procedures.

Genomic DNA was purified by proteinase K digestion of 5 μm sticks cut from paraffin sections. When matched normal tissue was not available, this was obtained by microdissection. Mutations and polymorphisms in p53 were identified by direct sequencing of exons individually amplified with commercially available Pfx DNA polymerase, in accordance with standard procedures.

χ² tests and Fisher's exact tests were used to test the frequencies of mutations, LOH and response to treatment rates between the different codon 72 genotypes. Survival times were calculated as the date of diagnosis to the date of death or the date of censor if alive. Progression-free survival times were calculated as the date of diagnosis to the earlier of date of progression or death, or date of censor if not progressed or died. Survival curves were constructed using the method of Kaplan and Meier, J. Amer. Statisticians Assoc., 53:457 (1958). The log-rank test (see Peto, et al., Br. J. Cancer, 35:1-39 (1977)) was used to assess the prognostic ability of retention of wild-type p53 alleles. Statistical analysis was carried out using a commercially available software program.

In the 43 patients whose cancers retained a wild-type p53 allele and who had an evaluable response to treatment, complete response appeared significantly linked with whether a cancer retained a wild-type 72R p53 allele, wild-type 72P p53 allele or both (P<0.04). Individuals whose cancers retained a wild-type 72R allele had the highest complete response rate (27 out of 28 patients, 96%). Both overall survival (OS) and progression-free survival (PFS) were significantly longer in patients whose cancers retained a wild-type p53 allele (either 72R or 72P), than in those lacking a wild-type allele (both P<0.0001). Furthermore, there was significantly different OS and PFS between cases retaining a wild-type 72R allele, cases retaining a wild-type 72P allele and cases retaining both wild-type 72R and 72P alleles (P=0.02 and 0.007, respectively), with the best prognosis in cases retaining a wild-type 72R allele.

### EXAMPLE 11

In these experiments, the steady state levels of endogenous p73 were analyzed via Western Blotting following drug treatment. To elaborate, various squamous carcinoma cancer cell lines, and Saos-2 p53^{-/-} cells were cultured in DMEM, with 10% fetal bovine system. The squamous carcinoma cell lines (“SCC”) tested included cells of the head and neck, cervix, vulva, and immortalized skin keratocytes. The cells were exposed to cytotoxic concentrations of drugs, i.e., 10 μM etoposide, 1 μg/ml and 10 μg/ml cisplatin, 500 ng/ml taxol, and 1 μg/ml doxorubicin. The cells were exposed to the drugs for 24 hours, after which they were harvested in a lysis buffer (50 mM Tris, 250 mM NaCl, 0.1% Nonidet NP-40, 5 mM EDTA, 1 mM PMSF, with a protease inhibitor cocktail). An affinity purified, p73 specific antibody was diluted 1:100, and used in Western blotting. In brief, equal amounts of protein, as determined by the Bradford method, were loaded onto lanes, following preclearance with protein A-Sepharose, and immunoprecipitation with the anti-p73 antibody. The immunoprecipitates were then resolved, on 10% polyacrylamide gels, and immunoblotted with the antibody, and a secondary anti-mouse light chain antibody conjugated to horseradish peroxidase, which was then visualized using standard methods.

The results indicated doxorubicin increased p73 in all cell lines. Head and neck squamous cell lines HSC3, and BICR31 showed an increase in p73 levels for all drugs tested. Doxorubicin taxol and cisplatin, but not etoposide, also increased the levels of p73 in head and neck squamous cell line HN30.

### EXAMPLE 12

In these experiments, quantitative PCR was carried out to measure expression of AIP1, in accordance with Keiser, et al., Proc. Natl. Acad. Sci. USA, 99:95-100 (2002), incorporated by reference. In brief, total RNA was prepared using commercially available products. The data indicated that AIP1 mRNA levels increased, resulting from the drugs that caused the upregulation of p73.

### EXAMPLE 13

In these experiments, the possibility that induction of p73 contributes to the cytotoxicity of the tested drugs was studied.

Saos-2 cells were transiently transfected, with one of pcDNA3HAp73α, which encodes full length p73α, and with pcDNA3p73Δ2 and pcDNA3p73DD, both of which encode dominant-negative inhibitors, or with pcDNAp73DD (L371P), which encodes an inactive point mutation. The Saos-2 cells were used because Costanzo, et al., Mol. Cell, 9:175-186 (2002), have shown that these activate an endogenous program of p73 dependent gene expression, in response to doxorubicin, including upregulation of AIP1, and are thus suitable for experiments of this nature.

The Saos-2 cells were maintained, as described, supra, and cell lines stably expressing the dominant negative inhibitors were generated by transfection, and then cultivation in the presence of 400 μg/ml of G418. Isolated colonies were ring cloned, and then the expression of transfected sequences was confirmed.

The cells, when in exponential growth phase, received 10 μg of a plasmid encoding p73, and 3 μg of pcDNA3CD20. Twenty-four hours after transfection, cells were harvested, stained with propidiumiodide, and apoptosis was determined as percentage of sub-G1 cells.

Both p73Δ2 and p73DD were found to bind to p73, and inhibit its transactivating activity whereas p73DD (L317P) locks this activity. See, e.g., Irwin, et al., Cancer Cell, 3:403-410 (2003); Fillipovich, et al., Oncogene, 20:514-522 (2001). Expression of p⁷³a by itself caused apoptosis, but the apoptosis was blocked by p73Δ2 and p73DD, but not P73DD (L371P).

The Saos-2 cells which expressed p73Δ2 and p73DD were less sensitive to apoptosis following exposure to cisplatin, doxorubicin, and taxol, but the lines expressing p73DD (L371P) exhibited sensitivity similar to control Saos-2 cells. In colony forming assays, the survival of lines which expressed either the dominant-negative form of p73 was higher than control cells, and those expressing inactive p73DD (L371P). In brief, this was determined by seeding 10²-10⁶ cells, in quadruplicate, onto 9 cm tissue culture dishes, and were treated, 24 hours later, for 2 hours, with cytotoxic drugs at various concentrations. Following drug exposure, the cells were grown until visible colonies appeared. They were then fixed in methanol, and stained with Giemsa. Survival was calculated relative to the cells receiving the drug vehicle alone.

### EXAMPLE 14

The preceding examples demonstrated a role for p73 in mediating cytotoxicity of anticancer agents. They show that cellular sensitivity can be modified by dominant negative inhibition of p73.

It has been recognized that mutants of p53 can inhibit p73 for mutants 143A and 175H. Marin, et al., Nat. Genet., 25:47-54 (2000), showed that this ability to inhibit p73 is enhanced if the mutant is expressed on the 72R polymorphic variant, together with a 143A, or 175H mutation. Given this background, a series of p53 mutations were tested for their ability to influence cellular sensitivity to p73 dependent, drug induced apoptosis in vitro.

In a first step, Saos-2 cells were transiently transfected with plasmids expressing p73α (10 μg), and a p53 mutant expressed with either 72P or 72R for each mutant (20 μg). The cells also received 3 μg of pCD20. Twenty-four hours later, cell cycle distribution, and DNA content, were analyzed, as described supra.

A total of 25 mutants were tested, and 13 produced at least 50% inhibition of p73 dependent apoptosis, i.e., 173L, 175H, 176Y, 179R, 179Y, 220C, 252Y, 242Y, 245D, 245S, 248W, 249S, 273C, and 282W. The 72R form of mutants inhibited apoptosis more efficiently.

### EXAMPLE 15

Given the data, supra, experiments were then carried out to determine if the observed inhibition correlated with the ability of the mutants to form stable complexes with p73.

A subset of the mutants were tested, in transfection/communoprecipitation assays, along the lines of those described by DiComo, et al., Mol. Cell Biol., 19:1438-1449 (1999); Marin, et al., supra, Strano, et al., J. Biol. Chem., 275:29503-29572 (2000), and Gaiddon, et al., Mol. Cell. Biol., 21:1874-1887 (2001), which were developed to show association between mutant p53 and p73.

Saos-2 cells were transfected with plasmids encoding HA tagged, p73α (10 μg), and 20 μg of a p53 mutant, in both the 72R and 72P polymorphic forms (175H, 173L, 281G, 179Y, 245D, 142L). After twenty-four hours, lysates were prepared, as discussed, supra, and precipitated with antibody PAb1801. Immunoblotting was then carried out with anti-HA antiserum.

The 281G mutations associated inefficiently with p73, and served as a negative control. The 142L mutant associated very weakly. All of the others, which had inhibited p73 dependent apoptosis, showed greater association with p73 in the 72R polymorphs, as compared to 72P.

### EXAMPLE 16

These experiments were designed to assess the effect of the p53 polymorphism on drug-induced apoptosis and cytotoxicity directly.

To do this, recombinant isogenic Saos-2 cells were prepared which expressed either the 72R or 72P polymorphism in each of the following mutations: 173L, 175H, 179Y, 142L. Hence, a total of eight recombinant lines were prepared, using the protocols for stable integration described supra. Cell lysates were prepared from exponentially growing cells, and subjected to Western blotting using a p53 specific antibody and an antibody to pDNA to verify equal protein loading in each lane.

The steady state levels of the p53 mutants in the Saos-2 recombinant lines were similar to endogenous mutant levels, as seen in head and neck cancer cell lines.

The cells were then exposed to various anticancer drugs, as described herein, and apoptosis was assessed via flow cytometry, as described supra. In brief, cells were contacted with 10 μg/ml of cisplatin for 2 hours, and then grown in drug free medium for 24 hours. Then, flow cytometry was carried out.

As compared to control cells, the 173L, 175H and 179Y mutants showed reduced sensitivity to drug induced apoptosis, for both polymorphic forms, as compared to controls.

It was noted that in cells expressing 72R-p53, apoptosis was reduced to nearly the levels of untreated controls. In contrast, reduction in apoptosis in the 72P form was not as pronounced.

Mutant 142L forms had only minimal effects on drug toxicity, which correlates with their low levels of association with p73.

These experiments were then extended to 72R and 72P variants of an additional 22 mutant forms. Of these, 176Y, 179R, 220C, 242Y, 245S, 245D, 282W, 249S, and 273C showed greater inhibition of apoptosis in the 72R form, as compared to 72P, while 142L, 152Q, 158G, and 161V had minimal effect.

### EXAMPLE 17

In these experiments, a subset of mutants were analyzed, to determine if differences in apoptotic inhibition were related to differences in ability to block induction of AIP1, which is recognized as a target of p73. See Costanzo, et al., Mol. Cell., 9:175-186 (2002). Saos-2 cell lines expressing the p53 mutants were transfected with AIP1 luciferase and pSV2-βgal. Cells were transfected with 1 μg of AIP1 luciferase and 2 μg of pSV2 β-galactosidase using commercially available products. Eighteen hours later, cells were treated with cisplatin (10 μg/ml) for 2 hours. Luciferase was determined after 12 hours. The 173L, 175H and 179Y mutants all inhibited activation of the AIP1 promoter, and the 72R isoform mutants blocked it more strongly than did the 72P isoforms. The 173L, 175H and 179Y mutants all inhibited activation of the AIP1 promoter by cisplatin and the 72R isoform mutants blocked it more strongly than did the 72P isoform mutants.

### EXAMPLE 18

In a set of follow up experiments, the effect of the mutations on anticancer drug cytotoxicity was tested, as described supra, using varying concentrations of drug and determining colony survival, as described supra.

The mutants which were shown, previously, to most efficiently inhibit apoptosis also conferred the highest degree of cytoxic resistance to cisplatin, i.e., 72R variants expressing mutants 173L, 175H, and 179Y.

### EXAMPLE 19

The prior examples suggested that mutant p53 increases resistance to cancer agents, via interaction with p73. To investigate this small inhibitory RNA (“siRNA”) oligonucleotides were used. The sequences:

| | |
|---|---|
| CUACUUCCUGAAAACAACG(d)TT | (SEQ ID NO: 1) |
| and | |
| CGUUGUUUUCAGGAAGUAGd(TT) | (SEQ ID NO: 2) |

were used to inhibit p73, and as a control to inhibit pGL3.

The siRNA oligos were transfected into cells, in amounts ranging from 10-100 μM, using commercially available reagents. The siRNA was introduced into cells expressing either the 72P 173L mutant or the 72R173L mutant. Both were shown, supra, to inhibit p73. A dose dependent reduction in steady state levels of p53 polymorphic variants of the mutant was shown following the siRNA treatment. The PGL3 control siRNA did not effect the levels.

In follow up experiments, the siRNA was used to test whether it could downregulate expression of the endogenous proteins in squamous carcinoma cell lines that had been shown to upregulate p73, in response to cytotoxic drug exposure.

The results indicated that the p53 siRNA reduced steady state levels of p53 in head and neck squamous cells, as well as other cells tested.

Cytotoxic drug treatment, as shown supra, did not increase p53 levels but when siRNA was added mutant p53 levels clearly dropped, both in the presence and absence of cytotoxic drugs. In brief, head and neck squamous cells were treated with 100 nM of either the p53 siRNA, or the PGL3siRNA, and were treated, 48 hours later, with doxorubicin (1 μg/ml), taxol (500 ng/ml), cisplatin (10 μg/ml), or were untreated. After 24 hours, cell lysates were prepared and analyzed via Western blotting. The p53 levels were unaffected by the drugs, but downregulated by siRNA.

### EXAMPLE 20

As a follow up to the work, supra, experiments were carried out to determine if the siRNA influenced association of the mutant p53 molecules with p73. Hacat cells received 100 nM of p53siRNA, or pGL3siRNA, and were either treated or not, 48 hours later, with 1 μg/ml doxyrubicin, 500 ng/ml taxol, 10 μg/ml cisplatin, or 10 μM etoposide. Cell lysates were prepared after 24 hours, and p53-p73 complexes were analyzed, as described supra.

The results indicated that the increase in p73 production associated with drug exposure was not affected by p53 siRNA, but there was a clear decrease in the amount of p53 which associated with p73.

Further, the cells which received siRNA for p53 and were then exposed to anticancer drugs showed a clear increase in apoptosis, as compared to controls.

Whereas there was variance in magnitude of enhancement of the apoptosis caused by mutant p53 downregulation, there was an increase in drug-induced apoptosis in all cell lines examined.

### EXAMPLE 21

The Saos-2 cells which had been engineered to stably express 72R and 72P mutants were then tested, in assays designed to determine whether p53 siRNA impacted drug resistance. Saos-2 cells which expressed 72R173Lp53 were treated with 100 nM of siRNA for p53, or with the same amount of PGL siRNA. After 48 hours, the cells were exposed to varying concentrations, of either cisplatin or doxyrubicin. Survival was assessed via colony forming ability.

Pretreatment with p53 siRNA rendered cells more sensitive to the cytotoxic drugs than any of the controls. When tested on the 143L mutant, which had been shown, previously, to have only a small impact on p73 inhibition, the siRNA had a similarly small effect.

When additional mutants were tested, it was found that siRNA for p53 increased sensitivity of cell lines expressing 175H, 179Y, 179R, 245S, 248W, 249S, 273C, and 282W, in both polymorphic forms, to both cisplatin and doxyrubicin.

### EXAMPLE 22

It can be concluded, from the experiments supra, that both p73 and p53 polymorphism have roles in influencing anticancer drug sensitivity. These experiments were carried out to determine whether treatment outcome in vivo is influenced by mutation and p53 polymorphism, in cancers where the tissues express p73.

To test this, the expression of p73, and the sequence of p53 were determined, in a series of 70 patients having inoperable head and neck cancer, who had received chemotherapy. Tissue samples were obtained as paraffin sections at diagnosis, or were fresh frozen samples. The patients had received a combined of treatment radiotherapy, and one of cisplatin/5-fluorouracil, these two drugs plus paclitoxel, or cisplatin and gemcitabine.

Genomic DNA was isolated from blood, via commercial methods, as was the genomic DNA from frozen tissue.

Codon 72 genotypes did not differ in frequency from normal tissue controls, i.e., RR:54%; RP:40%; PP:6%, while controls were RR:55%; RP:50%; PP:5% (χ²:1.85, p=0.40). Of 70 patient examined, 40 showed mutation.

There were more mutations in RP patients (19) in the R allele (14) than in the P allele (5).

Of 27 patients with wild type p53, 25 were fully responsive to treatment, while only 23 of 40 patients with mutations did so. Thirty of the patients had died when the genomic analysis was carried out, with median time to death or progression was 13.1 months. Median follow up for the 40 alive, progression free patients, was 33.9 months.

Progression free survival was significantly longer for the complete responders, than partial responders, i.e., 13 of 48 died or progressed, for complete responders, as compared to 14/19 for partial responders. A total of 77% survived progression free at two years, compared to 28%. Further, progression free survival was longer for wild type p53 patients than those with mutant p53, i.e., 5 of 30 progressed or died, as compared to 25 of 40 with mutations, with survival at two years 82% versus 46%.

For the 40 mutations, the progression free survival period was significantly longer for patients with mutated 72P, as compared to mutated 72R (1/8 versus 24/32 progressed or died; 2 year survival rate: 83% versus 38%).

For the 40 cases with mutations, the percentage survival at 2 years of 72P allele carriers was 100% (0/8), compared to 53% (21/32), for 72R.

### EXAMPLE 23

As a final set of experiments, the clinical effect of the mutations was assessed via studying clinical outcome of head and neck cancer patients whose cancers presented p53 mutations.

There were a total of 25 such cases, where p53 mutations were shown to inhibit p73, using the assays described supra, and 15 which did not. Of these 25, 11 achieved complete regression, and of the 15, 12 did. The group of 15 had significantly longer progression free survival with non p73⁻ inactivating mutations, as compared to the group of 25, 6/15 progressed or died, versus 19/25 progressed or died.

Of the 25 patients with mutations that inactivate p73, 6 were in the P allele, and 19 in the R allele. Of the mutated P allele, 5 of 6 achieved complete regression, and 6 of the 19 with mutations in the R allele did so. While 19 of the patients had died at the time of analysis, the progression free survival was much longer in patients with the mutation in the P allele (1/8 progressed or died), as compared to the R allele (18/19 progressed or died).

These data suggest p73 has an important role in mediating the activity of anticancer drugs. As such, experiments were carried out to determine if expression of p73 per se is of prognostic value. The expression of p53 and p73 was assessed in 50 cases, using immunocytochemistry. Of these, p53 was found in 31/50 cases, and p73 in 19/50. The patients were then grouped, into p73⁺, wt p53⁺(n=10), p73⁺, mut p53+(n=9), p73⁻wt p53⁺(n=11), and p73⁻ mut p53⁺(n=20).

Survival was found to be similar between the groups, suggesting p73 expression is not predictive of survival; however, it appears that the combination of p73 expression and p73 mutational status, taken together, is predictive of response.

The foregoing examples establish a feature of the invention, which is a link between expression of p73 and mutated forms of p53, wherein expression of these two molecules is indicative of decreased susceptibility to chemotherapy, i.e., a poor prognosis for the subject. The data indicate that mutations at any position other than the normally occurring polymorphism at position 72 is indicative of this poor prognosis.

The data demonstrate that p73 status per se influences responsiveness to cytotoxic, anti-cancer drugs. It was shown, e.g., that p73 is induced in response to anticancer agents in squamous cell lines. The upregulation is accompanied, inter alia, by increased expression of AIP1. The diversity of anticancer drugs which cause p73 induction and AIP1 upregulation support the hypothesis that p73 may be an important cellular mediator of the cytotoxicity of multiple anticancer agents.

The data, supra show that abrogation of p73 function via expression of dominant-negative inhibitors reduced apoptosis and cytotoxicity. Hence, a feature of the invention also relates to a method for treating a subject who expresses p73 and a mutated form of p53, so as to inhibit or “override” the effect of the mutant. This therapeutic approach may involve, e.g., the administration of a p53 inhibitor, such as a small molecule, an antibody, or siRNA, but may also involve the administration of p73, in an amount sufficient to override the impact of the mutated p53. The administration may involve, e.g., the use of the p73 protein per se, as well as gene therapy methods, where a nucleic acid molecule encoding multiple copies of p73 is introduced to the subject.

It is also known that the expression of p73 is deregulated via E2F1, when oncogenes are expressed. See, e.g., Irwin, et al., Nature, 407:645-648 (2000); Stiewe, et al., Nat. Genet., 276:464-469 (2000); Brooks, et al., Br. J. Cancer, 86:263-268 (2002); Zarka, et al., J. Biol. Chem., 26:11310-11316 (2001). Full-length p73 is overexpressed in some cancers, relative to normal tissues, such as head and neck squamous cell carcinoma. Hence, another therapeutic approach relies on inhibition of E2F1, so that the deregulation of p73 is blocked.

Yet another feature of the invention relates to the ability to assess susceptibility to cytotoxic drug therapy via assaying for the particular polymorphic form of p53 being expressed by the patient. As was shown, supra, the prognosis for subject patients with the 72R non-mutated polymorphic form is better than that of subjects who express the non-mutated, 72P polymorphic variant. Hence, a further feature of the invention is a method for diagnosing a subject to determine susceptibility to drug therapy by assaying for the non-mutated 72P or 72R form. This can be done via, e.g., nucleic acid analysis, such as PCR or other hybridization methodologies, or protein analysis, such as sequence determination, immunoassay, etc.

It is also envisioned that a subject's therapeutic “profile” may be improved, via-administering the 72R polymorphic form of p53 to subjects in need thereof, such as those who express 72P, or mutated forms of p53, regardless of the allelic form. This 72R can be administered as a protein, but also via gene therapy.

The mechanism explicated herein also allows the skilled artisan to screen compounds to determine their usefulness as therapeutic agents. To elaborate, as a link has been shown between apoptosis and the particular form of p53 present, one may assess a particular compound's potential efficacy by carrying out an in vitro assay on cells which have been prescreened to determine the form of p53 present, be it wild type p53 72R or 72P, or a mutated form, and determine whether or not the compound affects the rate at which the cell is driven to apoptosis. Conversely, potential carcinogens can be assessed the same way, i.e., by determining if the compound inhibit apoptosis. In the case of known anti-cancer agents, the efficacy of new drug combinations can be assessed in the same way.

Other features of the invention will be clear to the skilled artisan, and need not be reiterated here.

1. Method for determining susceptibility of a cancer patient to chemotherapy comprising determining if cancer cells of said patient express wild type p53 with arginine at position 72 or wild type p53 with proline at position 72, and wherein proline at position 72 is indicative of poor susceptibility to chemotherapy, and arginine at position 72 is indicative of susceptibility greater than that where position 72 is proline.

2. The method of claim 1, comprising determining if the cancer cells of said patient express wild type p53 with arginine at position 72 or wild type p53 with proline at position 72 via PCR or immunoassay.

3. (canceled)

4. The method of claim 1, wherein said cancer is a squamous cell cancer.

5. (canceled)

6. A method for improving susceptibility of a subject to chemotherapy wherein said subject suffers from cancer, and whose cancer cells express wild type p53 with proline at position 72, comprising administering to said subject an amount of wild type p53 with arginine at position 72 sufficient to augment said chemotherapy.

7. The method of claim 6, wherein said subject has received or is receiving anticancer therapy.

8. The method of claim 7, wherein said subject received or is receiving chemotherapy therapy.

9. The method of claim 6, further comprising adding wild type p53 which enhances apoptotic activity in said cancer cell.

10. Method for determining susceptibility of a cancer patient to chemotherapy comprising determining if cancer cells of said patient expresses p73 and determining if said patient expresses a mutated form of p53, wherein expression of both p73 and a mutated form of p53 is indicative of decreased susceptibility to chemotherapy.

11. The method of claim 10, wherein said mutated form is a mutated form of wild type p53 having arginine at position 72 or proline at position 72.

12. (canceled)

13. The method of claim 10, comprising determining if the cancer cells of said patient express p73 and said mutated form of p53 via PCR or immunoassay.

14. (canceled)

15. The method of claim 10, wherein said cancer is squamous cell cancer.

16. (canceled)

17. A method for reducing drug resistance in a cancer patient, whose cancer cells express p73 and a mutated form of p53 comprising administering to said patient an inhibitor of mutated p53-p73 interaction in an amount sufficient to inhibit said interaction.

18. The method of claim 17, wherein said mutated form is a mutated form of wild type p53 having arginine at position 72 or proline at position 72

19. (canceled)

20. The method of claim 17, wherein said cancer is head and neck cancer.

21. The method of claim 17, wherein said inhibitor is RNAi, an antisense molecule, or an antibody which binds specifically to a form of p53.

22. A method for screening a compound for cancer causing or cancer inhibiting properties, comprising contacting a cell which expresses a mutated form of wild type p53 with arginine at position 72 or proline at position 72, determining impact of said compound on apoptosis of said cell, and comparing it to apoptosis of a cell expressing said mutant which has not been contacted with said compound, a difference therebetween indicating that said compound is cancer causing or cancer inhibiting.

23. A method for determining susceptibility to chemotherapy comprising assaying a sample of cells from a patient for expression of wild type p53 with arginine at position 72 and a mutation at position 46, wherein presence of arginine at position 72 and a mutation at position 46 are indicative of increase susceptibility to chemotherapy as compared to cells which do not have a mutation at position 46.

24. The method of claim 23, wherein said chemotherapy comprises administration of an apoptosis inducing agent.

25. The method of claim 23, wherein said mutation is a change from Ala to Ser.


SEQUENCE LISTING (2 sequences)

SEQ ID NO: 1 - RNA, 21 nt, Artificial sequence
  misc_RNA: small inhibitory RNA
cuacuuccugaaaacaacguu

SEQ ID NO: 2 - RNA, 21 nt, Artificial sequence
  misc_RNA: small inhibitory RNA
cguuguuuucaggaaguaguu